(19) United States
(12) Patent Application Publication
(10) Pub. No.: US 2026/0115346 A1
(43) Pub. Date: Apr. 30, 2026

(54) METHOD AND DEVICE FOR DISINFECTING AND/OR STERILISING OBJECTS

(71) Applicant: SCHWING TECHNOLOGIES GMBH, Neukirchen-Vluyn (DE)

(72) Inventor: Alfred SCHILLERT, Dorsten (DE)

(21) Appl. No.: 19/117,012

(22) PCT Filed: Sep. 29, 2023
(86) PCT No.: PCT/EP2023/077149

§ 371 Date: Mar. 28, 2025

(30) Foreign Application Priority Data
Sep. 30, 2022 (DE) 10 2022 125 305.8

(51) Int. Cl. A61L 2/24; A61L 2/18; A61L 101/36; A61L 103/15; A61L 103/25; A61L 103/55; A61L 103/65; G01N 33/00

(52) U.S. Cl. CPC A61L 2/24; A61L 2/18; G01N 33/0027; A61L 2101/36; A61L 2103/15; A61L 2103/25; A61L 2103/55; A61L 2103/65; A61L 2202/122; A61L 2202/123; A61L 2202/13; A61L 2202/14; A61L 2202/15; A61L 2202/181

(57) ABSTRACT

The invention relates to a method for disinfecting and/or sterilizing objects, said method comprising the method steps of: placing an object in a process chamber that can be evacuated; evacuating the process chamber; introducing a treatment medium; carrying out the disinfection and/or sterilization process; and increasing the pressure in the process chamber, wherein the concentration of the treatment medium is monitored while the disinfection and/or sterilization process is carried out. The invention also relates to: a software program for carrying out the method for disinfecting and/or sterilizing objects; and a device for disinfecting and/or sterilizing objects. The invention also relates to: a disinfected object comprising residues of the treatment medium and/or reaction products of the treatment medium and fewer than 2000 germs per m² on the surface of the disinfected object; and a sterilized object comprising residues of the treatment medium and/or reaction products of the treatment medium and a germ-free surface.

The invention relates to a method for disinfecting and/or sterilizing objects, comprising the steps of placing an object in a process chamber that can be evacuated, evacuating the process chamber, introducing a treatment medium, carrying out the disinfection and/or sterilization process and increasing the pressure in the process chamber, wherein the concentration of the treatment medium is monitored while the disinfection and/or sterilization process are carried out. The invention also relates to a software program for carrying out the method for disinfecting and/or sterilizing objects and to a device for disinfecting and/or sterilizing objects. The invention also relates to a disinfected object having residues of the treatment medium and/or reaction products of the treatment medium and less than 2000 germs per m² on the surface of the disinfected object, and to a sterilized object having residues of the treatment medium and/or reaction products of the treatment medium and a germ-free surface.

### PRIOR ART

The peracetic acid-ethanol vacuum method is an established and cost-effective sterilization method for allogeneic bone transplants and also offers adequate antibacterial, antifungal and antiviral protection for allogeneic soft tissue transplants.

In the case of disinfection and/or sterilization by the use of PES at reduced pressure, the PES passes into the gas phase at a high reduced pressure already at a low temperature. This process is used to achieve the best possible disinfection and/or sterilization of the products at a harmless temperature.

Introduced in the 1980s [Starke R, Hackensellner HA, von Versen R. Experimentelle Untersuchungen zur Entkeimung von Transplantationsmaterial mit Peressigsäure. Z Exp Chir Transplant Artificial organs. 1984; 17 (5): 254-8.], more than 60,000 allogeneic bone grafts have been sterilized with peracetic acid (PES, CH3CO3H) and ethanol to date [131]. Several studies have demonstrated sufficient efficacy and safety of the procedure and have not demonstrated an adverse effect on the structural and biomechanical properties of the transplants [132, 133]. Viral protection was also confirmed by the sterilization of soft tissue transplants such as Achilles tendon, cartilage and skin [152]. Furthermore, Scheffler et al. were not able to detect any impairment of the structural and material properties of human BPTB allografts after sterilization with the peracetic acid-ethanol negative pressure method in an in vitro study [153]. In addition, PES showed no cytotoxic or pro-inflammatory effect on human patellar tendon allografts in another in vitro study [98. In this publication, the advantages of the peracetic acid/ethanol vacuum process are used for disinfecting and/or sterilizing objects, for example trousers or gloves.

### Object

It is therefore an object of the present invention to provide a method for disinfecting and/or sterilizing objects, with which objects can be disinfected and/or sterilized reliably and cost-effectively.

It is also an object of the present invention to provide a method for disinfecting and/or sterilizing objects, with which objects can be disinfected and/or sterilized reliably and cost-effectively.

It is also an object of the present invention to provide a disinfected object.

It is also an object of the present invention to provide a sterilized object.

The object is achieved with the method for disinfecting and/or sterilizing objects according to claim 1. Advantageous embodiments of the invention are provided in the dependent claims.

The method according to the invention for disinfecting and/or sterilizing objects has five method steps: In the first step, an object is placed on a receptacle in a process chamber that can be evacuated of a device for disinfecting and/or sterilizing. For the purposes of this specification, an object is a component which optionally has internal surfaces. An object is, for example, a garment, in particular for use in laboratories and/or medical facilities. An object is therefore, for example, a pair of trousers, a jacket, a hood or a glove. The process chamber can be evacuated and can be opened or closed in a gas-tight manner. The volume of the process chamber can be variably selected depending on the dimensions of the object to be disinfected and/or sterilized. Placement of an object can be done automatically, remotely and/or manually by a user. The process chamber can have suitable receptacles for this purpose.

The difference between a disinfection and a sterilization is that in a sterilizing treatment of an object all microorganisms, including their permanent forms (spores), are killed or irreversibly inactivated. The object is then generally free of microorganisms capable of propagation, and at most one of 10⁶ germs must survive, i.e. only one of 1,000,000 treated objects would not be sterile. In a disinfecting treatment of an object, pathogenic germs are likewise killed or irreversibly inactivated, but the reference to the number of germs to be eliminated is smaller by a power of 10 than in the case of sterilization, the aim of the disinfection is to reduce the germs by at least a factor of 10⁵.

In the second process step, the process chamber is evacuated. A vacuum of less than 300 mbar, preferably less than 150 mbar, particularly preferably less than 50 mbar is preferably produced in the process chamber.

In the third process step, a treatment medium is introduced. For the purposes of this document, a treatment medium is peracetic acid or a peracetic acid-containing mixture having a peracetic acid content of at least 2% by volume, preferably at least 3% by volume and particularly preferably at least 4% by volume of peracetic acid. Alternatively, the starting substances of the peracetic acid can also be introduced into the process chamber in order to allow the peracetic acid to be formed in situ.

In the fourth step, the disinfection and/or sterilization process is carried out. For this purpose, the process parameters (pressure and temperature in the process chamber) are kept constant during an exposure time. This is to eliminate germs, bacteria and viruses on the object. Only the pressure is increased by the vaporization of the PES. Optionally, the pressure change can be avoided by appropriate pumping out.

In the fifth step, the pressure in the process chamber is increased. When the pressure is increased, the process chamber contains a concentration of the treatment medium which is below the concentration of the treatment medium in the process chamber during the holding time of the process. The concentration of the treatment medium during or after the increase of the pressure in the process chamber is below the concentration of the treatment medium during the holding time by more than a factor of 10, preferably by more than a factor of 30.

According to the invention, the concentration of the treatment medium is monitored during the disinfection and/or sterilization process. For carrying out the disinfection and/or sterilization process, different concentrations of the treatment medium are provided in order to be able to flexibly design the disinfection and/or sterilization process. Depending on the type and dimensions of the object, different concentrations of the treatment medium are necessary to ensure thorough disinfection and/or sterilization. For this purpose, it is necessary to monitor the concentration of the treatment medium in the process chamber during the disinfection and/or sterilization process. The device for disinfection and/or sterilization has suitable sensors for this purpose.

In a further development of the invention, the concentration of the treatment medium in the chamber is monitored. The device for disinfection and/or sterilization has suitable sensors for this purpose.

In a further embodiment of the invention, the device for disinfection and/or sterilization has a sensor unit with a sensor, the concentration of the treatment medium being measured with the sensor. The sensor is optionally a pressure sensor which detects the internal pressure of the sample chamber. The sensor can also be a chemical sensor which detects the concentration of a chemical substance to be determined.

In a further embodiment of the invention, the sensor is arranged in the sample chamber. Depending on the type and dimensions of the object, different concentrations of the treatment medium are necessary to ensure thorough disinfection and/or sterilization. For this purpose, it is necessary to monitor the concentration of the treatment medium in the process chamber during the disinfection and/or sterilization process.

In a further embodiment of the invention, the sample chamber has an outlet and a medium discharge for discharging the treatment medium from the sample chamber, wherein a sensor is arranged outside the sample chamber after the outlet in the medium discharge. The sensor in the media outlet detects the concentration of the treatment medium in the outlet. As a result, the consumption of the treatment medium during the disinfection and/or sterilization process can be detected and it can also be determined whether the disinfection and/or sterilization process has been completed. If the concentration of the treatment medium in the outlet is low, the disinfection and/or sterilization process is not yet complete, and if necessary the concentration of the treatment medium in the sample chamber must be increased. At a high concentration of the treatment medium in the outlet, which reaches the initial concentration of the treatment medium in the sample chamber, the disinfection and/or sterilization process is completed.

In a further development of the invention, a sensor of the sensor unit detects chemical measurement variables. In a further development of the invention, the sensor is a gas sensor. In particular, the sensor detects the concentration of one or more definable gases.

In a further embodiment of the invention, a sensor of the sensor unit detects physical measurement data. The sensor detects physical variables such as optionally a pressure and/or temperature in the sample chamber.

In a further embodiment of the invention, the device for disinfection and/or sterilization has a control which is suitable for this purpose and/or is provided for carrying out a control program which controls the process parameters of the method for disinfection and/or sterilization. The control is designed as a microcontroller with memory, which has a software program for controlling a method for disinfecting and/or sterilizing objects. In order to carry out the method, the method is controlled as a function of the measured variables detected by the sensors of a sensor unit.

In a further embodiment of the invention, the control is coupled to the sensor unit, a pump, an inlet and/or the outlet. The control is designed as a microcontroller with memory, which has a software program for controlling a method for disinfecting and/or sterilizing objects. In order to carry out the method, the pump, the inlet and/or the outlet are controlled as a function of the measured variables detected by the sensors of a sensor unit.

In a further embodiment of the invention, the treatment medium is introduced as a function of the determined concentration of the treatment medium. The process chamber has a suitable sensor for determining the concentration. By introducing the treatment medium as a function of its concentration in the process chamber, it is ensured that the concentration of the treatment medium is always correct in order to achieve rapid and thorough disinfection and/or sterilization of the object.

In a further development of the invention, the process chamber is preheated. In a further embodiment of the invention, the process chamber is preheated to the desired temperature of the process of 35° C.-85° C., preferably 40° C.-75° C. and particularly preferably 50° C.-65° C. The method for disinfecting and/or sterilizing objects is thus accelerated.

In a further embodiment of the invention, the objects are packaged in sterilization bags before being placed in the process chamber. In a further aspect of the invention, the sterilization bags are permeable to vapors. Sterilization bags, also known as autoclave bags or laminated bags, are used to protect items from contamination. They are made of a material that withstands high temperatures and steam sterilization. This makes them ideal for use in hospitals, dental practices and other medical facilities. Sterilization bags provide a safe and efficient way to protect the object from contamination. With their puncture-resistant construction and moisture-resistant material, they are ideal for use in any environment. Sterilization bags are puncture-resistant so that they are not damaged during the sterilization process. The material of the sterilization bag is moisture and bacteria resistant, so that the objects are protected from contamination. The sterilization bags are easy and simple to store so that they can be used in any environment.

In a further embodiment of the invention, the process chamber is closed after the object has been placed in the process chamber. After closing, the process chamber is sealed in a gas-tight manner.

In a further embodiment of the invention, the process chamber is heated to process temperature after the process chamber has been closed. Due to the placement of the object, the temperature in the process chamber decreases, so a renewed heating up to the process temperature of 40° C.-65° C., preferably 50° C.-55° C., takes place.

In a further embodiment of the invention, the pressure in the process chamber is monitored. For this purpose, the process chamber is connected to a pressure sensor.

In an advantageous embodiment of the invention, the treatment medium is a PES solution. PES disinfects chemically and has an oxidizing effect on microorganisms. It has a broad spectrum of action, short exposure time and an irreversible effect. In addition, compared to other sterilization media, it is largely compatible with materials, can be added in exact amounts and, because of the low concentration used (dilution with water), is slightly to not damaging to the skin. Optionally, a mixture of peroxyacetic acid, acetic acid, a strong oxidizing medium such as, for example, hydrogen peroxide and water is used. The acids and hydrogen peroxide are powerful oxidizing mediums that decompose microorganisms.

In a further embodiment of the invention, the process chamber is evacuated to a pressure of 1-50 mbar, preferably 10 mbar. In a further embodiment of the invention, the pressure in the process chamber is maintained below the boiling curve of the treatment medium after the introduction of the treatment medium. In a further development of the invention, the pressure is kept below the boiling curve of the treatment medium throughout the process duration of the disinfection and/or sterilization process. After the treatment medium has been introduced, the treatment medium passes immediately into the gas phase. As a result, even regions of the object which are difficult to access, in particular internal surfaces of the object, are disinfected and/or sterilized.

In a further embodiment of the invention, after completion of the disinfection and/or sterilization process, the process chamber is flushed with ambient air, the flushing preferably being carried out at intervals. This ensures that the treatment medium has been completely removed from the process chamber and/or the object and cannot escape uncontrollably when the process chamber is opened, so that the operator or a bystander does not inhale the solution when the door or access is opened. For the purposes of this specification, a ventilation medium is air and technical gases. In a further aspect of the invention, ambient air up to a chamber pressure of 200-500 mbar is introduced during flushing.

In a further embodiment of the invention, after introduction of the ambient air up to a pressure of 200-500 mbar, the process chamber is evacuated again to 1-100 mbar, preferably 10-40 mbar, particularly preferably 20 mbar. This likewise ensures that the treatment medium has been completely removed from the process chamber and cannot escape uncontrolled when the process chamber is opened.

In a further embodiment of the invention, the process steps according to claims 25 and 26 are repeated once, preferably three times and particularly preferably five times. This likewise ensures that the treatment medium has been completely removed from the process chamber and cannot escape in an uncontrolled manner when the process chamber is opened.

In a further embodiment of the invention, the pressure in the process chamber is increased to ambient pressure after completion of the flushing process. The disinfected objects can then be removed from the process chamber.

In a further development of the invention, the treatment medium is discharged. The treatment medium is thus removed from the process chamber.

In a further embodiment of the invention, a neutralizing medium is introduced in order to neutralize the treatment medium. PES is an acid, therefore a base is optionally used as the neutralization medium. This likewise ensures that the treatment medium has been completely removed from the process chamber and cannot escape uncontrolled when the process chamber is opened.

The object is likewise achieved by means of the software program for carrying out the method according to one or more of claims 1 to 29.

The software program according to the invention is suitable for carrying out the method for controlling processes by means of defined values and/or courses related to pressure, injection quantities (with defined concentrations), temperature and PES concentration in the chamber and by means of material and/or product information, their quantity, size, shape, etc., their degree of contamination and loading and/or type (spores, viruses, etc.). The software program uses the hardware of the device for disinfecting and/or sterilizing objects.

The object is also achieved by means of the device for disinfecting and/or sterilizing objects. Advantageous embodiments of the invention are provided in the dependent claims.

The device according to the invention for disinfecting and/or sterilizing objects has a process chamber that can be evacuated. The process chamber can be evacuated and can be opened or closed in a gas-tight manner. The volume of the process chamber can be variably selected depending on the dimensions of the object to be disinfected and/or sterilized.

The device also has a first connection and/or first reservoir for a treatment medium and a first inlet in the process chamber, wherein the first inlet is connected to a first connection and/or the first reservoir for the treatment medium. The treatment medium is preferably peracetic acid or a peracetic acid-containing mixture having a peracetic acid content of at least 2% by volume, preferably at least 3% by volume and particularly preferably at least 4% by volume of peracetic acid. Alternatively, the starting substances of the peracetic acid can also be introduced into the process chamber in order to allow the peracetic acid to be formed in situ.

The device likewise has a second connection and/or second reservoir for a ventilation medium and a second inlet in the process chamber, the second inlet being connected to the second connection and/or the second reservoir for the ventilation medium and/or to the first connection and/or the first reservoir. The ventilation medium is preferably air; technical gases, e.g. noble gases, are also possible.

Furthermore, the device has a pump, which is connected to one or more outlets, and a control for controlling the first inlet, the second inlet, the first connection, the second connection and/or the outlet. Optionally, the temperature variation and/or the pressure and/or the injection quantity and/or the concentration of a treatment medium in the chamber is controlled by the control. Furthermore, the duration and the number of process phases and cycles can be controlled by the control.

The device further comprises a sensor unit. Depending on the type and dimensions of the object, different concentrations of the treatment medium are necessary to ensure thorough disinfection and/or sterilization. For this purpose, it is necessary to monitor the concentration of the treatment medium in the process chamber during the disinfection and/or sterilization process. The device for disinfection and/or sterilization has a sensor unit with suitable sensors for this purpose. Optionally, a sensor of the sensor unit detects chemical measurement variables. In particular, the sensor detects the concentration of one or more definable gases. Optionally, a further sensor of the sensor unit detects physical measurement data. The sensor detects physical variables such as pressure and/or temperature in the sample chamber and/or the treatment medium.

The concentration of peracetic acid in the gas phase can be measured by various methods: One possibility is to determine the peracetic acid concentration by means of the structure of an electrochemical cell. Sensors constructed in this way use the current produced during the reduction of peracetic acid to calculate the concentration in conjunction with the Nernst equation. The peracetic acid is hereby reduced to acetic acid. However, a sensor can also be constructed on the basis of spectroscopic methods. It is known to determine the absorption bands of peracetic acid and the influence of acetic acid and hydrogen peroxide on the spectra by spectroscopic methods. Both measurements in the infrared range and measurements in the UV range are possible. In the UV range, for example, a measurement at 340 nm in vacuo is suitable for the measurement of peracetic acid, in the infrared range, inter alia, a band at 1295 cm⁻¹.

In a further embodiment of the invention, the device has a medium discharge for discharging the treatment medium from the chamber. The media discharge is a tubular conduit connected to the sample chamber. Optionally, the medium discharge is connected to a filter and a pump via which the treatment medium can be discharged from the process chamber.

In a further embodiment of the invention, the control is coupled to the sensor unit, a pump, the first inlet, the second inlet and/or the outlet. The control is designed as a microcontroller with memory, which has a software program for controlling a method for disinfecting and/or sterilizing objects. In order to carry out the method, the pump, the inlet and/or the outlet are controlled as a function of the measured variables detected by the sensors of a sensor unit.

In a further embodiment of the invention, the sensor unit has a sensor. The sensor optionally detects physical variables such as pressure and/or temperature in the sample chamber and/or the treatment medium. The sensor can also be a chemical sensor which detects the concentration of a chemical substance to be determined.

In a further development of the invention, the sensor is a pressure sensor. In a further aspect of the invention, the pressure sensor is arranged in the sample chamber. The pressure sensor detects the pressure in the sample chamber and/or the treatment medium.

In a further embodiment of the invention, a sensor is a chemical sensor which is suitable and/or provided for detecting a chemical measured variable. The chemical sensor detects the concentration of a chemical substance to be determined.

In a further embodiment of the invention, a sensor is a chemical sensor which is suitable and/or provided for detecting a physical measured variable from which the concentration of the treatment medium in the chamber can be determined. Depending on the type and dimensions of the object, different concentrations of the treatment medium are necessary to ensure thorough disinfection and/or sterilization. For this purpose, it is necessary to monitor the concentration of the treatment medium in the process chamber during the disinfection and/or sterilization process.

In a further embodiment of the invention, the chemical sensor is arranged in the chamber or in the medium discharge for discharging the treatment medium from the chamber. The sensor in the medium discharge detects the concentration of the treatment medium in the outlet. As a result, the consumption of the treatment medium during the disinfection and/or sterilization process can be detected and it can also be determined whether the disinfection and/or sterilization process has been completed. If the concentration of the treatment medium in the outlet is low, the disinfection and/or sterilization process is not yet complete, and if necessary the concentration of the treatment medium in the sample chamber must be increased. At a high concentration of the treatment medium in the outlet, which reaches the initial concentration of the treatment medium in the sample chamber, the disinfection and/or sterilization process is completed.

The object is also achieved by means of the disinfected object. Advantageous embodiments of the invention are provided in the dependent claims.

The disinfected object has a surface roughness Rz with Rz<=10 μm, preferably with Rz<=5 μm and particularly preferably with Rz<=1 μm and/or with a porosity with a pore size P with 10 nm<=P<=500 μm, preferably with 25 nm<=P<=100 μm and particularly preferably with 50 nm<=P<=50 μm and/or a pore volume V with V>=70%, preferably V>=55% and particularly preferably V>=40%, based on the total volume of the object. The disinfected object also has residues of the treatment medium and/or reaction products of the treatment medium of less than 2000 germs per m² on the surface of the disinfected object. The disinfected object according to the invention has a contamination with germs on its surface of less than 2000 germs per m², preferably of less than 1000 germs per m² and particularly preferably of less than 500 germs per m², which corresponds to a germ reduction of 84% to 99.9%. In a further development of the invention, the disinfected object has residues of peracetic acid or residues of reaction products of peracetic acid.

The values for roughness Rz mentioned in this document were determined by means of the stylus method (see DIN EN ISO 25178-601). The porosity values mentioned in this document with the pore volume and pore size parameters were determined by means of mercury porosimetry in accordance with DIN ISO 15901-1, the information on pore size relating to the average pore size.

In a further aspect of the invention, the porosity is an open porosity, i.e. the object has cavities which are connected to one another and to the environment. The disinfected object is an object from the following groups: masks, goggles/visors, surgical textiles, disposable protective gowns, lightweight hoods (fan filter system), fan filter devices, protective overalls (rescue service/fire brigade), surgical shoes, magnifying-glass surgery, respirator hoses, masks, bags, pari-boy nebulizers for inhalation therapy (with electronics), fever thermometers, full-face masks, lung machines, compressed air breathers, but also electronic devices such as radios and a smartphone. The material of the disinfected object is: PP, PET/PETG, nylon, PU/PU foams, PVC foam, ABS, elastane, EPDM, silicones, rubber or metal, or a composite of the aforementioned materials.

The object is also achieved by means of the sterilized object. Advantageous embodiments of the invention are provided in the dependent claims.

The sterilized object has a surface roughness Rz with Rz<=10 μm, preferably with Rz<=5 μm and particularly preferably with Rz<=1 μm and/or with a porosity with a pore size P with 10 nm<=P<=500 μm, preferably with 25 nm<=P<=100 μm and particularly preferably with 50 nm<=P<=50 μm and/or a pore volume V with V>=70%, preferably V>=55% and particularly preferably V>=40%, based on the total volume of the object. The sterilized object also has residues of the treatment medium and/or reaction products of the treatment medium and a germ-free surface.

The sterilized object according to the invention has a contamination with germs on its surface of 0 if the probability of a surviving germ is less than 1:1,000,000 (<=10⁻⁶ per unit of the object).

In a further development of the invention, the sterilized object has residues of peracetic acid or residues of reaction products of peracetic acid. In an optional further development, the concentration of the treatment medium and/or of reaction products of the treatment medium is below 0.1 ml/m² or below 0.32 mg/m2 and, in a particularly preferred embodiment, below 0.05 ml/m² or below 0.16 mg/m²

In a further aspect of the invention, the porosity is an open porosity, i.e. the object has cavities which are connected to one another and to the environment. The sterilized object is an object from the following groups: masks, goggles/visors, surgical textiles, disposable protective gowns, lightweight hoods (fan filter system), fan filter devices, protective overalls (rescue service/fire brigade), surgical shoes, magnifying-glass surgery, respirator hoses, masks, bags, pari-boy nebulizers for inhalation therapy (with electronics), fever thermometers, full-face masks, lung machines, compressed air breathers, but also electronic devices such as radios and a smartphone. The material of the sterilized object is: PP, PET/PETG, nylon, PU/PU foams, PVC foam, ABS, elastane, EPDM, silicones, rubber or metal, or a composite of the aforementioned materials.

Exemplary embodiments of the device and method according to the invention for disinfecting and/or sterilizing objects are shown schematically in the drawings in a simplified manner and are explained in more detail in the following description.

In the drawings:

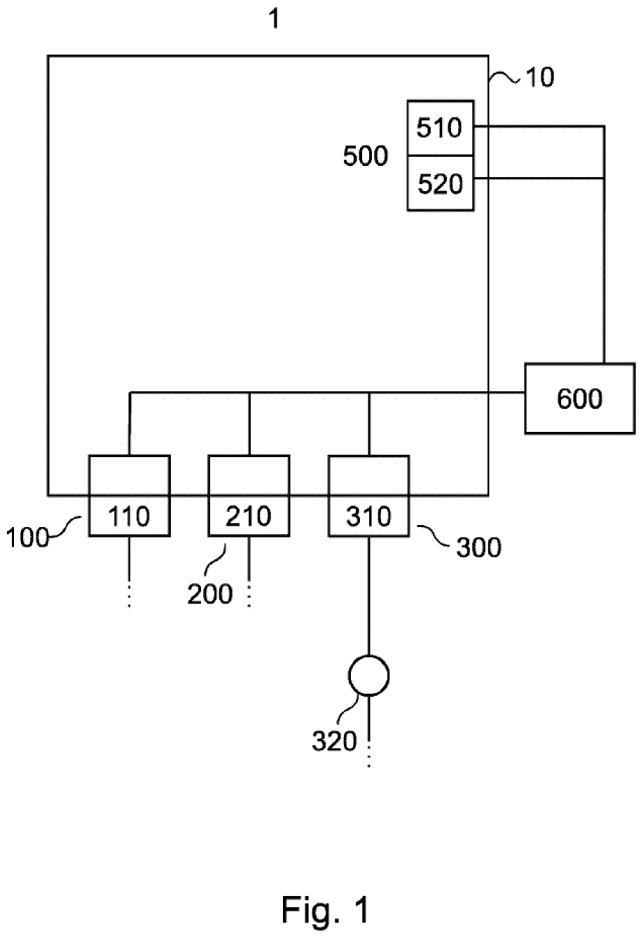

FIG. 1 shows a device for disinfecting and/or sterilizing objects, sensor unit in process chamber

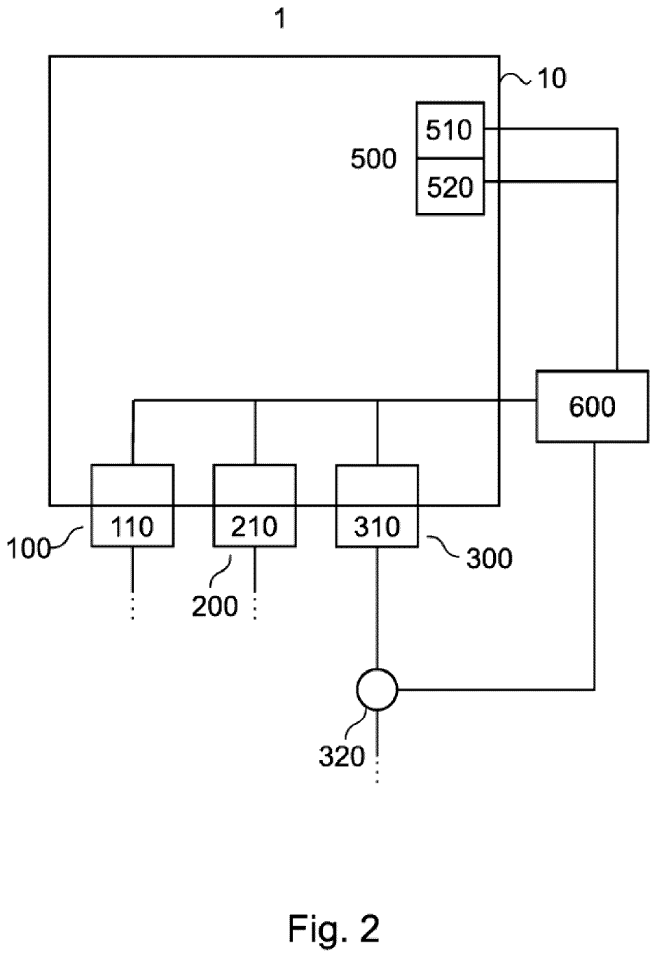

FIG. 2 shows a device for disinfecting and/or sterilizing objects, sensor unit in process chamber, vacuum pump connected to control

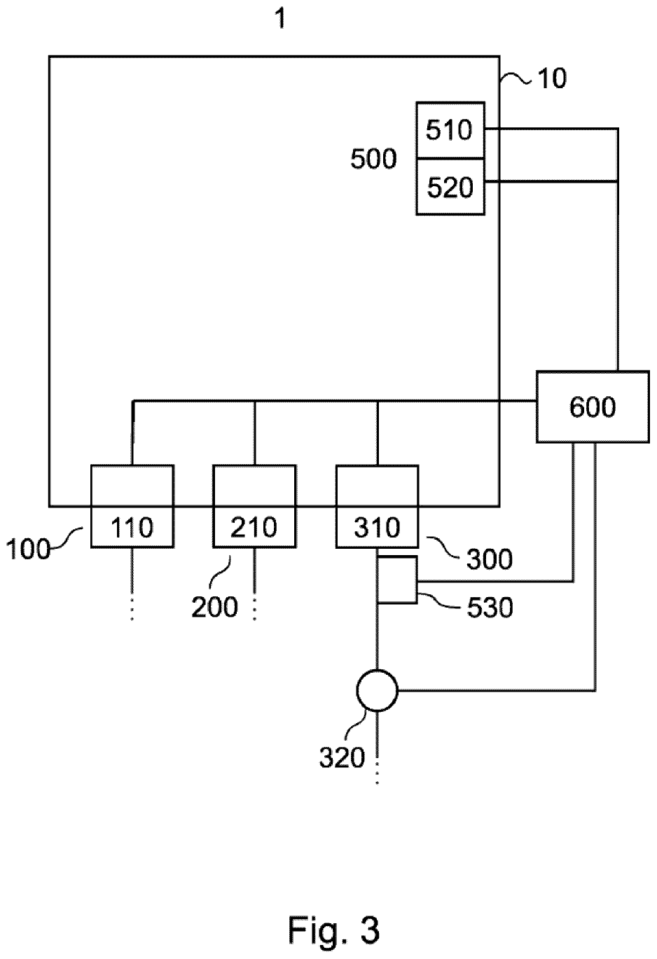

FIG. 3 shows a device for disinfecting and/or sterilizing objects, sensor unit in process chamber, vacuum pump connected to control, chemical sensor in medium discharge

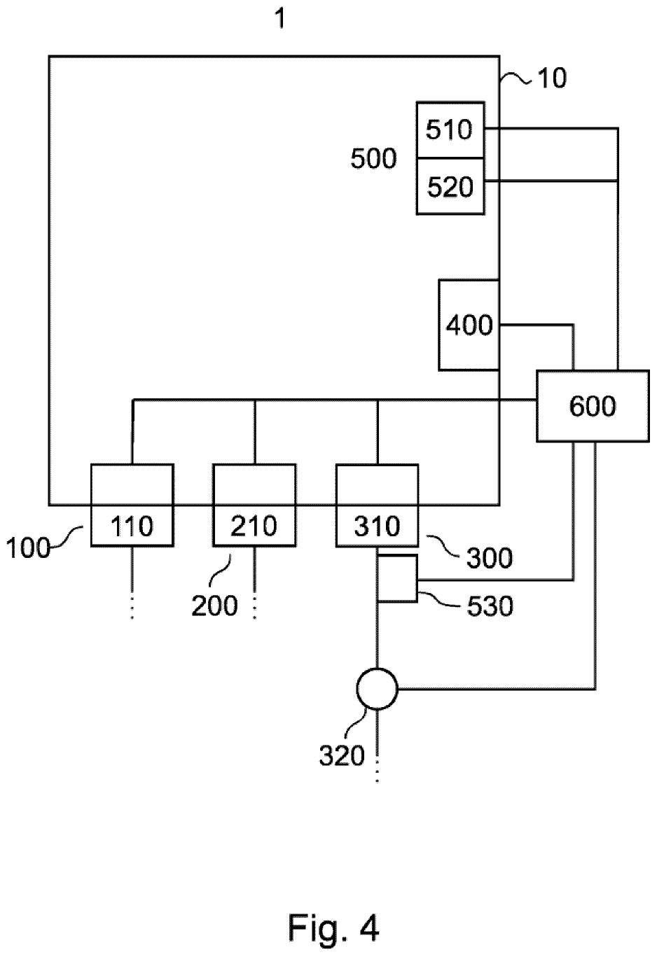

FIG. 4 shows a device for disinfecting and/or sterilizing objects, sensor unit in process chamber, vacuum pump connected to control, chemical sensor in medium discharge, heating device in process chamber

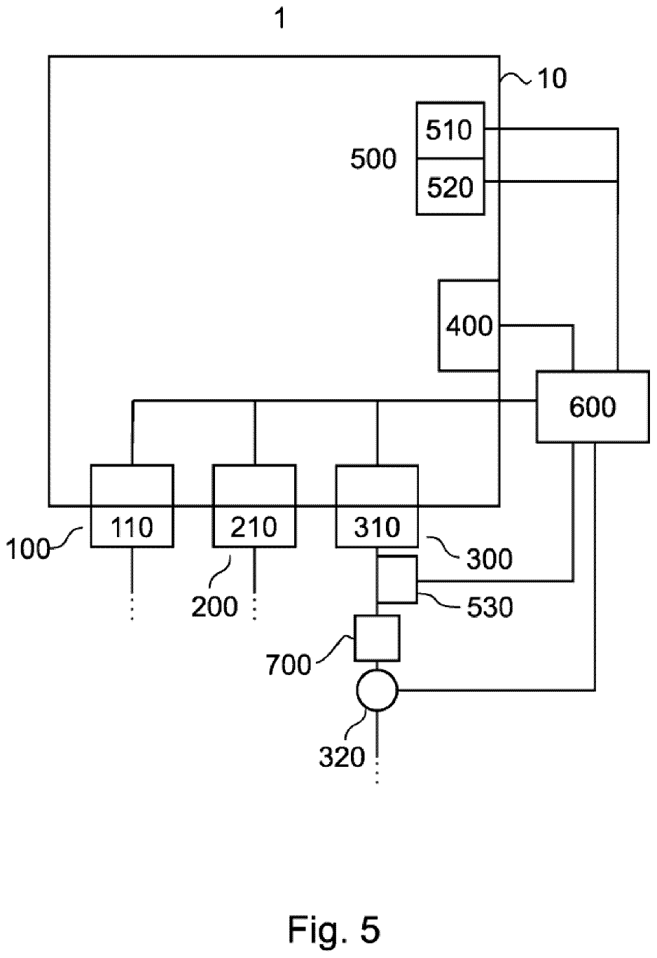

FIG. 5 shows a device for disinfecting and/or sterilizing objects, sensor unit in process chamber, vacuum pump connected to control, chemical sensor and HEPA filter in medium discharge, heating device in process chamber

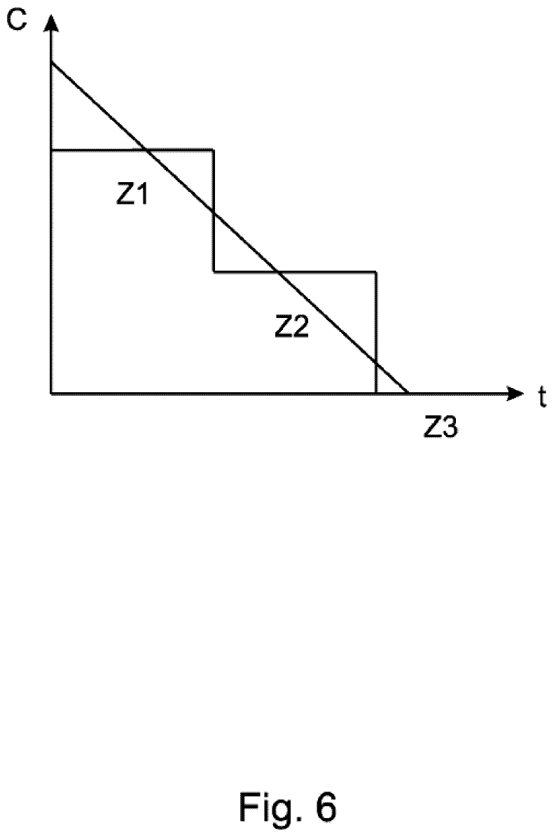

FIG. 6 shows an exemplary embodiment of the method for disinfecting and/or sterilizing objects

FIG. 1 and FIG. 2 show exemplary embodiments of the device 1 according to the invention for disinfecting and/or sterilizing objects. The device 1 has the process chamber 10, which is designed to be evacuable and has a volume of 50 liters, and 100 liters are also possible. For evacuation, the vacuum pump 320, designed as a rotary pump, is connected via the outlet connection 310 to the outlet 300 of the process chamber 10 (FIG. 1). The vacuum pump 320 is connected to the control 600 (FIG. 2). The outlet 300 can be opened or closed and is connected to the control 600. The device 1 has the medium discharge 330 for discharging the treatment medium from the process chamber 10.

In addition to a UI, the control 600 has a microcontroller and a memory which has a suitable software program for carrying out a disinfection and/or sterilization process of object 2. The process chamber 10 has a first inlet 100 which is connected via a first connection 110 to a reservoir for the treatment medium. The first inlet 100 is connected to the control 600. The second inlet 200, which is connected via the second connection 210 to the reservoir for the ventilation medium, is also connected to the control 600. The inlets 100, 200, like the outlet 300, can each be opened or closed via valves, controlled by the control 600. In addition, a sensor unit 500 with a temperature sensor 510 and a pressure sensor 520 is arranged in the process chamber 10 and is likewise connected to the control 600

For disinfection and/or sterilization of objects 2, the object 2 is placed in the process chamber 10, for example in a suitable receptacle in the process chamber 10. The object 2 is preferably packed in a vapor-permeable sterilization bag before it is placed in the process chamber 10, in order not to contaminate the object 2 during and after the disinfection and/or sterilization and to be able to place the contaminated object simply and safely in the process chamber 10.

The process chamber 10 is then sealed in a gas-tight manner and evacuated to 20 mbar by means of the vacuum pump 320. The treatment medium is then introduced by the control 600 sending a signal for opening the first inlet 100 to the first inlet 100. The first inlet 200 is connected to the reservoir for the treatment medium. In all the exemplary embodiments shown here, the treatment medium is a mixture of peroxyacetic acid and water and, if appropriate, other constituents. The treatment medium is preferably peracetic acid or a peracetic acid-containing mixture having a peracetic acid content of at least 2% by volume, preferably at least 3% by volume and particularly preferably at least 4% by volume of peracetic acid. Alternatively, the starting substances of the peracetic acid can also be introduced into the process chamber in order to allow the peracetic acid to be formed in situ. Due to the low pressure in the process chamber 10, the solution is brought into the gaseous state in order to fill the process chamber 10 and carry out the oxidation process on the objects to be disinfected. Different concentrations of PES are provided for the process in order to be able to flexibly design the disinfection and/or sterilization process. The first inlet 100 is closed again after introduction of the treatment medium.

For disinfection and/or sterilization of the objects 2, the object 2 is left in the process chamber 10 for between 5 minutes and 120 minutes at constant pressure and temperature in the process chamber 10 and exposed to the PES atmosphere. Thereafter, the pressure in the process chamber 10 is increased by the control 600 sending a signal for opening the second inlet 200 to the second inlet 200. The second inlet 200 is connected to the reservoir for the treatment medium. In all exemplary embodiments, the ventilation medium is air, and technical gases, for example noble gases, are also possible. When the pressure is increased, the process chamber 10 contains a concentration of the treatment medium which is below the concentration of the treatment medium in the process chamber 10 during the holding time of the process. The concentration of the treatment medium during or after the increase of the pressure in the process chamber 10 is below the concentration of the treatment medium during the holding time by more than a factor of 10, preferably by more than a factor of 30. The process chamber 10 can be opened and the object 2 removed.

FIG. 3 shows a further exemplary embodiment of the device 1 according to the invention for disinfecting and/or sterilizing objects 2. The device 1 shown here corresponds to that in the preceding exemplary embodiment (cf. FIG. 2), only the medium discharge 330 for discharging the treatment medium from the process chamber 10 has a sensor 530 which is connected to the control 600.

The sensors 510, 520 are arranged in the process chamber 10 and detect physical measured values in the process chamber 10. The sensor 510 is a temperature sensor for detecting and monitoring the temperature within the process chamber 10. The sensor 520 is a pressure sensor for detecting and monitoring the pressure within the process chamber 10. The sensor 530 arranged outside the process chamber 10 in the medium outlet 330 is a chemical sensor for detecting and monitoring the concentration of the treatment medium (PES) in the medium outlet 330. Alternatively, the sensor 530 is arranged in a bypass to the process chamber (10).

FIGS. 4 and 5 show further exemplary embodiments of the device 1 according to the invention for disinfecting and/or sterilizing objects 2. The devices 1 shown here corresponds to that in the preceding exemplary embodiment (cf. FIG. 3), only the process chamber 10 has a heating device 400, which is designed as a resistance heating and is connected to the control 600 (FIG. 4). In a further embodiment, the device 1 additionally has a HEPA filter 700 which is arranged in the line between pump 320 and outlet 300 (FIG. 5).

For the disinfection and/or sterilization of objects 2, after placing the object 2 in the process chamber 10 and evacuating it, the temperature in the process chamber 10 is increased by means of the heating device 400 after closing the process chamber 10, to a temperature of 50° C. to 55° C., or alternatively to a temperature of 40° C. to 65° C. In order to shorten the heating process, the process chamber 10 can be preheated before the object 2 is placed in the process chamber 10.

In the next step, the treatment medium (PES solution) is introduced into the process chamber 10 via the first inlet 100. In an advantageous further development of the invention, the injection quantity and/or the concentration of the treatment medium in the process chamber 10 is controlled with the aid of the control 600. Furthermore, the duration and the number of process phases and cycles are controlled. The PES solution begins to boil immediately and the pressure in the process chamber 10 increases. During part of the process, the control 600 controls the pressure and temperature in the process chamber 10 such that the boiling curve of the PES solution is not exceeded in order to prevent the PES solution from condensing out. This state is kept constant for part of the process duration. This is to eliminate germs, bacteria, and viruses on object 2. Only the pressure in the process chamber 10 is increased by the evaporation of the PES. Optionally, this pressure change can be avoided by appropriate pumping out by means of the pump 320.

By means of the temperature sensor 510, the control 600 continuously detects the temperature in the process chamber 10 during the process and controls the temperature in the process chamber 10 via the heating device 400. The pressure in the process chamber 10 is detected by the pressure sensor 520 and is likewise regulated by the control 600, in that either the inlet 100 is opened or the pressure in the chamber is reduced via the outlet 300 and the pump 320. By means of the chemical sensor 530, the concentration of the treatment medium (PES solution) in the medium outlet 330 is continuously detected and monitored by the control 600. For this purpose, the outlet connection 310 is constantly open during the execution of the method.

As a result, the consumption of the treatment medium during the disinfection and/or sterilization process can be detected and it can also be determined whether the disinfection and/or sterilization process has been completed. If the concentration of the treatment medium in the medium discharge 330 is low, the disinfection and/or sterilization process is not yet complete, and if necessary the concentration of the treatment medium in the sample chamber 10 must be increased. For this purpose, the first inlet 100 is opened by means of the inlet connection 110 under the control of the control 600 and further treatment medium is conducted into the sample chamber 10. At a high concentration of the treatment medium in the medium discharge 330, which reaches the initial concentration of the treatment medium in the sample chamber, the disinfection and/or sterilization process is completed.

After the end of the process, the ventilation medium (air) is flushed through the process chamber 10 at intervals. The ventilation medium is preferably germ-free, i.e. no microorganisms and/or viruses which are greater than 0.45 micrometer, preferably 0.22 micrometer, are contained in the ventilation medium. For this purpose, ambient air up to a pressure in the process chamber 10 of from 200 mbar to 500 mbar, preferably 300 mbar, is allowed into the process chamber 10 via the second inlet 200 under the control of the control 600. It is then pumped off to 1 mbar to 100 mbar, preferably about 40 mbar, particularly preferably about 20 mbar. This flushing process is carried out one to five times, preferably three times, in order to ensure that the treatment medium has been completely removed from the process chamber 10, cannot escape uncontrollably when the process chamber 10 is opened, and the operator or a bystander does not inhale the solution when the door or the access is opened. In a further development, the ventilation medium is introduced until the concentration of the treatment medium in the chamber is between 0.5 g/m and 4 g/m³; preferably between 1 g/m³ and 3 g/m³; particularly preferably between 1.5 g/m³ and 2.5 g/m³. The air inlet or the air discharge via an outlet 300 and the medium discharge 330 are preferably effected via HEPA filters. The HEPA filter 700 serves to protect the environment and persons from germs during the generation of vacuum at the beginning of the process and also to protect the object 2 from contamination.

FIG. 6 shows an exemplary embodiment of the method according to the invention for disinfecting and/or sterilizing objects 2, which is carried out in three cycles. The graph shows the concentration of spores/viruses/bacteria C on the inner and outer surfaces of the object 2 (ordinate) over time t (abscissa).

After carrying out the method with the device 1 according to the invention, the object 2 has traces of the treatment medium (PES) and/or of reaction products of the treatment medium with other substances on its surface. In an optional further development, the concentration of the treatment medium and/or of reaction products of the treatment medium is below 0.1 ml/m² or below 0.32 mg/m² and, in a particularly preferred embodiment, below 0.05 ml/m² or below 0.16 mg/m²

After carrying out the method with the device 1 according to the invention, the disinfected object 2 according to the invention has a contamination with germs on its surface of less than 2000 germs per m², preferably of less than 1000 germs per m² and particularly preferably of less than 500 germs per m² of surface, which corresponds to a germ reduction of 84% to 99.9%. The sterilized object 2 according to the invention has a contamination with germs on its surface of 0 if the probability of a surviving germ is less than 1:1,000,000 (<=10⁻⁶ per unit of the object 2).

The disinfected and/or sterilized object 2 has a surface roughness Rz with Rz<=10 μm, preferably with Rz<=5 μm and particularly preferably with Rz<=1 μm and/or with a porosity with a pore size P with 10 nm<=P<=500 μm, preferably with 25 nm<=P<=100 μm and particularly preferably with 50 nm<=P<=50 μm and/or a pore volume V with V>=700%, preferably V>=55% and particularly preferably V>=40%, based on the total volume of the object.

The method according to the invention which can be carried out by means of the device 1 according to the invention is so effective that also the inner surfaces of the object 2 protected by the splashproof housing after carrying out the method have such a low contamination with germs as the outer surfaces.

The disinfection and sterilization process is usually carried out depending on the object to be sterilized/disinfected, the framework conditions and predefinable requirements according to processes optimized especially in the control 600 and the respective sequence. For this purpose, different cycles, intervals and process parameters can be set via a software program stored in the control 600 or, if required, also individually via the UI of the control 600. For example, different cycles of disinfection and/or sterilization may have to be carried out at a high germ load of the object 2, since the PES solution is consumed at a high germ load, so that the subsequent addition of PES may be necessary. This step will take place, among other things, in cycles. During the process, the treatment medium (PES) is pumped out and added or ventilation is performed. In addition, the course of the cycle is also suitable for small volumes both of the process chamber 10 and of the object 2 as well as for difficult to reach and complex geometries of the object 2.

TABLE 1
Overview of validation tests for disinfection
of spore strips SAL 10⁵

| | | Concentration | | Biological |
|---|---|---|---|---|
| | PES in | of PES in | Process | result |
| | solution | process chamber | duration | #not sterile/ |
| | [%] | [g/m³] | [min] | #tested |
| 1 | 6 | 3 | 240 | 0/2 |
| 2 | | | 120 | 0/1 |
| 3 | | 1.7 | 150 | 0/2 |
| 4 | 4 | 2 | 120 | 0/1 |
| 5 | | 1.9 | 120 | 0/2 |
| 6 | | | 90 | 0/2 |
| 7 | | | 60 | 0/4 |
| 8 | | | 45 | 0/1 |
| 9 | | | 40 | 0/2 |
| 10 | | | 30 | 0/2 |
| 11 | | | 20 | 0/2 |
| 12 | | | 15 | 0/1 |
| 13 | | | 10 | 0/1 |
| 14 | | | 5 | 0/1 |
| 15 | | 1.7 | 20 | 0/1 |
| 16 | | | 15 | 0/1 |
| 17 | | 1.5 | 240 | 0/2 |
| 18 | | | 150 | 0/2 |
| 19 | | 1.3 | 20 | 0/1 |
| 20 | | | 15 | 0/1 |
| 21 | | 0.94 | 60 | 0/5 |
| 22 | | | 40 | 0/1 |
| 23 | | | 30 | 0/2 |
| 24 | | | 20 | 0/2 |
| 25 | | | 15 | 0/1 |
| 26 | | | 10 | 0/1 |
| 27 | | | 5 | 0/1 |
| 28 | 4 | 0.47 | 60 | 0/1 |
| 29 | | | 30 | 0/1 |
| 30 | | | 15 | 0/1 |
| 31 | | | 10 | 0/1 |
| 32 | | | 5 | 0/1 |

TABLE 2
Overview of validation tests for disinfection of laboratory
utensils dripped with spore suspension SAL 10⁶

| | | Concentration | | |
|---|---|---|---|---|
| | PES in | of PES in | Process | Biological result |
| | solution | process chamber | duration | #not sterile/ |
| | [%] | [g/m³] | [min] | #tested |
| 5 | 4 | 1.9 | 120 | 0/4 |
| 7 | | | 60 | 0/4 |
| 8 | | | 45 | 0/4 |
| 12 | | | 15 | 0/4 |
| 13 | | | 10 | 0/4 |
| 14 | | | 5 | 0/4 |
| 15 | | 1.7 | 20 | 0/4 |
| 16 | | | 15 | 0/4 |
| 19 | | 1.3 | 20 | 0/4 |
| 20 | | | 15 | 0/4 |
| 21 | | 0.94 | 60 | 0/4 |
| 22 | | | 40 | 0/4 |
| 23 | | | 30 | (pipette) 1/8 |
| 24 | | | 20 | 0/8 |
| 25 | | | 15 | (pipette) 1/4 |
| 26 | | | 10 | 0/4 |
| 27 | | | 5 | (pipette) 1/4 |
| 28 | 4 | 0.47 | 60 | 0/4 |
| 29 | | | 30 | 0/4 |
| 30 | | | 15 | (Eppi) 1/4 |
| 31 | | | 10 | (pipette) 1/4 |
| 32 | | | 5 | (mask, pipette) 2/4 |

In a disinfecting treatment of an object, pathogenic germs are also killed or irreversibly inactivated, but the reference to the number of germs to be eliminated is smaller by a power of 10 than in the case of sterilization, the aim of the disinfection is to reduce the germs by at least a factor of 10⁵. The disinfecting treatment then causes the object to cease to be infectious.

The efficiency of the disinfecting treatment is defined by the probability of the presence of microorganisms hostile to life. This probability is expressed by the sterility assurance level (SAL), a SAL of at least 10⁵ defines disinfection, a SAL of at least 10⁶ defines sterilization, i.e. the lower the SAL value, the higher the safety.

The disinfectant and sterilizing medium acting in the apparatus presented here is peracetic acid (PES) in a solution diluted with water. PES disinfects chemically and has an oxidizing effect on microorganisms and/or viruses It has a broad spectrum of action, short exposure time and an irreversible effect. In addition, compared to other sterilization media, it is largely compatible with materials, can be added in exact amounts and, because of the low concentration used (dilution with water), is slightly to not damaging to the skin.

The validation tests of the “disinfection and sterilization device” were carried out with spores of the bacterium Geobacillus stearothermophilis. Bacterial spores have a very high resistance (C) to chemical disinfection and/or sterilization. If bacterial spores can be successfully disinfected in the processes, it is to be assumed that the process also successfully disinfects microorganisms with moderate resistance (A) such as lipophilic viruses, vegetative bacteria, fungi (including spores), leading organisms such as E. faecium, S. aureus, P. aeruginosa, A. niger and microorganisms with high resistance (B) such as mycobacteria, hepatitis B virus and hydrophilic viruses such as the leading virus polio.

The validation experiments were first carried out with bio-indicator-spore strips 6×36 mm, on which in each case a colony-forming unit (CFU) of 10⁵ of the Geobacillus stearothermophilus was inoculated. The spore strips were each packaged in a sterilization bag. Different process times and different concentrations of PES were tested in the experiments. All tests were carried out at a chamber temperature of 50-55° C.

No subsequent growth or multiplication of the spores could be detected in all the spore strips tested at different PES concentrations and different process times.

In addition, untreated test samples were added to each procedure. This ensured that the samples were inoculated with spores.

In addition to the validation tests with the SAL 10⁵ spore strips, various disposable products were also drizzled with a spore suspension. These were a vaccine eye, a disposable 3 ml pipette, a microreaction vessel with a lid (Eppi) and a piece of nose-mask. The spore suspension was an alcoholic solution interspersed with Geobacillus stearothermophilus at a CFU/ml of at least 10⁶. Due to the small amount, the samples were each dripped with 2 drops of the suspension (in the pipette and the Eppi the suspension was dripped into this) and then placed in a sterilization bag. One drop corresponds to 0.05 ml, so that two drops correspond to a unit of 0.1 ml. Therefore the CFU per object is about 10⁵.

The samples were co-tested with some of the spore strip tests listed above. After the treatment, the swabs of the samples, analogous to the spore strips, were placed in a nutrient solution in the external laboratory for seven days and then examined for possible growth of the spores. In various experiments, spore growth was observed after seven days. This is due to the relatively low PES concentration in the process chamber as well as to the short process duration.

The disinfection or sterilization system according to the invention is designed in such a way that selectively different concentrations of PES can be set in the vacuum of the process chamber. By means of the combination of special injection technology, adapted vacuum process and process cycles, it is ensured that a distribution of the disinfectant and/or sterilizing medium mixture also takes place reliably at all inaccessible points and thus the PES has a very high activity against biogenic contamination even at low temperatures of 40° C. to 65° C., preferably 50° C. to 55° C.

The disinfection or sterilization system according to the invention, including the disinfection and/or sterilization methods and associated equipment technology, thus has the following decisive advantages: high efficiency, low-temperature processes (also suitable for thermolabile polymers), low operating costs, short process times, individual adaptations of the disinfection and/or sterilization programs to different requirements, very good scalability (from mobile desktop equipment to mobile room-sized systems) and general use of a reliable process which is not susceptible to malfunctions.

Due to the combination of the above advantages this innovative disinfection and sterilization system—in contrast to the methods already used—is therefore excellently suited for numerous known and new fields of application. For example, infection protection in small to large healthcare and care facilities, where the entire spectrum from compact desktop devices to large systems is required. The same applies to the areas of application of fire brigades and civil protection, in which the systems and equipment must be particularly robust and reliable as well as in some cases mobile.

In future, the PES processing system will include the following process-specific and application-related innovations in particular:
a. Novel, very widely usable low-temperature chemical disinfection and/or sterilization system.
The PES as reactive component can either be produced in low concentrations in the in-situ mixing of the different starting materials or can be adjusted to higher concentrations by selective mixing from a storage container in the system. PES reacts with the bacteria, viruses, spores, etc., which are killed quickly and efficiently. The individual components (hydrogen peroxide and acetic acid) for the in-situ production of PES are low-cost chemicals available worldwide, which can be handled safely while taking into account the necessary occupational safety requirements.
Gaseous PES is characterized by a high sterilizing effect. The use of gaseous PES in an automated vacuum process of a closed system ensures both a hazard-minimized handling and a safe effect
Standardized, validatable method (no manual disinfection, e.g. wipe disinfection)
The starting materials (hydrogen peroxide and acetic acid) can be removed quantitatively again from the treatment system and treated products very easily) so that the treated protective clothing can be reused without endangering the operators.
b. Mobile, tailor-made disinfection and/or sterilization device technology can be used for almost all application and use conditions for rapid PES disinfection and/or sterilization even on site in the event of major damage events.
c. Integrated monitoring and documentation system for the disinfection and/or sterilization process, i.e. for the protective effect after treatment of the PPE
d. Special design and process control of this chemical-physical treatment process (PES, vacuum, temperature) in combination with the specially adapted treatment system provides short treatment times of in some cases 8-25 minutes. These treatment times are dependent on the treatment objective (disinfection up to sterilization if necessary), the PPE materials or material combinations as well as the surfaces and geometries.
e. The disinfection and/or sterilization of the PPE is possible in the new preparation system in the sterilization bag and thus safe and contamination-free handling.

Various objects were treated with the method according to the invention in order to disinfect and sterilize them.

The following process parameters were used in the disinfection process:

| | Pressure | Concentration of PES | Process |
|---|---|---|---|
| Temperature | (process start) | in process chamber | duration |
| [° C.] | [mbar] | [g/m³] | [min] |
| 55 | 20 | 1.5 | 15 |

The following process parameters were used in the sterilization process:

| | Pressure | Concentration of PES | Process |
|---|---|---|---|
| Temperature | (process start) | in process chamber | duration |
| [° C.] | [mbar] | [g/m³] | [min] |
| 55 | 20 | 4 | 30 |

The treated objects are summarized in the following tables:

| | | Number of | Number of |
|---|---|---|---|
| | Roughness | germs/m³ after | germs/m³ after |
| Object | Rz in μm | disinfection | sterilization |
| Surgical clogs | 8.7 | 1370 | germ-free |
| disposable gloves | 4.35 | 920 | germ-free |
| visor | 0.85 | 360 | germ-free |

| | | Number of | Number of |
|---|---|---|---|
| | pore size | germs/m³ after | germs/m³ after |
| object | P in μm | disinfection | sterilization |
| blower | 12 nm- | 1740 | germ-free |
| filter unit | 430 μm | | |
| Infusomat | 18 nm- | 1160 | germ-free |
| | 92 μm | | |
| Mobile electronic | 32 nm- | 430 | germ-free |
| display device | 48 μm | | |

| | | Number of | Number of |
|---|---|---|---|
| | pore volume | germs/m³ after | germs/m³ after |
| object | V in % | disinfection | sterilization |
| FFP mask | 81 | 1220 | germ-free |
| MNS | 67 | 870 | germ-free |
| protective | 43 | 270 | germ-free |
| overalls | | | |

### LIST OF REFERENCE NUMERALS

1 device for disinfecting and/or sterilizing objects
10 process chamber
100 first inlet
110 first connection
200 second inlet
210 second connection
300 outlet
310 outlet connection
320 pump
330 medium discharge
400 heating device
500 sensor unit
510 temperature sensor
520 pressure sensor within
530 chemical sensor outside
600 control
700 HEPA filter
800 receptacle
2 object

1. A method for disinfecting and/or sterilizing objects (2) comprising the steps:
placing an object (2) in a process chamber (10) that can be evacuated, of a device (1) for disinfection and/or sterilization,
evacuating the process chamber (10),
introducing a treatment medium,
carrying out the disinfection and/or sterilization process,
increasing the pressure in the process chamber (10)
wherein the concentration of the treatment medium is monitored during the disinfection and/or sterilization process.

2. The method for disinfecting and/or sterilizing objects (2) according to claim 1,
characterized in that
the concentration of the treatment medium in the process chamber (10) is monitored.

3. The method for disinfecting and/or sterilizing objects (2) according to claim 1,
characterized in that
the device (1) for disinfection and/or sterilization has a sensor unit (500) with a sensor (520),
wherein the concentration of the treatment medium is measured with the sensor (520).

4. The method for disinfecting and/or sterilizing objects (2) according to claim 1,
characterized in that
the sensor (520) is arranged in the process chamber (10).

5. The method for disinfecting and/or sterilizing objects (2) according to claim 1,
characterized in that
the device (1) has an outlet (300) and a medium discharge (330) for discharging the treatment medium from the process chamber (10), wherein a sensor (530) is arranged outside the process chamber (10) downstream of the outlet (300) in the medium discharge (330) and/or in a bypass to the process chamber (10).

6. The method for disinfecting and/or sterilizing objects (2) according to claim 1,
characterized in that
a sensor (530) of the sensor unit (500) detects chemical measurement variables.

7. The method for disinfecting and/or sterilizing objects (2) according to claim 1,
characterized in that
the sensor (530) is a gas sensor.

8. The method for disinfecting and/or sterilizing objects (2) according to claim 1,
characterized in that
a sensor (510, 520) of the sensor unit (500) detects physical measurement data.

9. The method for disinfecting and/or sterilizing objects (2) according to claim 1,
characterized in that
the device (1) for disinfection and/or sterilization has a control (600) which is suitable for this purpose and/or is provided for carrying out a control program which controls the process parameters of the method for disinfection and/or sterilization.

10. The method for disinfecting and/or sterilizing objects (2) according to claim 1,
characterized in that
the control (600) is coupled to a sensor unit (500), a pump (320), the inlet (100) and/or an outlet (300),
wherein, in order to carry out the method, the pump (320), the inlet (100) and/or the outlet (300) are controlled as a function of the measured variables detected by the sensors of the sensor unit (500).

11. The method for disinfecting and/or sterilizing objects (2) according to claim 1,
characterized in that
the treatment medium is introduced as a function of the determined concentration of the treatment medium.

12. The method (400) for disinfecting and/or sterilizing objects according to claim 1,
characterized in that
the process chamber (10) is preheated.

13. The method for disinfecting and/or sterilizing objects (2) according to claim 1,
characterized in that
the process chamber (10) is preheated to the desired temperature of 40° C. to 65° C., preferably 50° C. to 55° C.

14. The method for disinfecting and/or sterilizing objects (2) according to claim 1,
characterized in that
the objects (2) are packaged in sterilization bags before the placement in the process chamber (10).

15. The method for disinfecting and/or sterilizing objects (2) according to claim 1,
characterized in that
the sterilization bags are permeable to vapors.

16. The method for disinfecting and/or sterilizing objects (2) according to claim 1,
characterized in that
the process chamber (10) is closed after the object (2) has been placed in the process chamber (10).

17. The method for disinfecting and/or sterilizing objects (2) according to claim 1,
characterized in that
the process chamber (10) is heated to process temperature after the process chamber (10) has been closed.

18. The method for disinfecting and/or sterilizing objects (2) according to claim 1,
characterized in that
the pressure in the process chamber (10) is monitored.

19. The method for disinfecting and/or sterilizing objects (2) according to claim 1,
characterized in that
the treatment medium is a PES solution.

20. The method for disinfecting and/or sterilizing objects (2) according to claim 1,
characterized in that
the process chamber (10) is evacuated to a pressure of 1-50 mbar, preferably 10 mbar.

21. The method for disinfecting and/or sterilizing objects (2) according to claim 1,
characterized in that
the pressure after introduction of the treatment medium is kept partly below the boiling curve of the treatment medium.

22. The method for disinfecting and/or sterilizing objects (2) according to claim 21,
characterized in that
the pressure is kept below the boiling curve of the treatment medium throughout the process and/or part of the process duration of the disinfection and/or sterilization process.

23. The method for disinfecting and/or sterilizing objects (2) according to claim 1,
characterized in that
after completion of the disinfection and/or sterilization process, the process chamber (10) is flushed with ambient air at intervals.

24. The method for disinfecting and/or sterilizing objects (2) according to claim 1,
characterized in that
ambient air up to a chamber pressure of 200-500 mbar is introduced during flushing.

25. The method for disinfecting and/or sterilizing objects (2) according to claim 1,
characterized in that
after introduction of the ambient air up to a pressure of 200-500 mbar, the process chamber (10) is evacuated again to 1-100 mbar, preferably 10-40 mbar, particularly preferably 20 mbar.

26. The method for disinfecting and/or sterilizing objects (2) according to claim 1,
characterized in that
ambient air up to a chamber pressure of 200-500 mbar is introduced during flushing and after introduction of the ambient air up to a pressure of 200-500 mbar, the process chamber (10) is evacuated again to 1-100 mbar, preferably 10-40 mbar, particularly preferably 20 mbar are repeated once, preferably three times and particularly preferably five times.

27. The method for disinfecting and/or sterilizing objects (2) according to claim 1,
characterized in that
after completion of the flushing process, the pressure in the process chamber (10) is increased to ambient pressure.

28. The method for disinfecting and/or sterilizing objects (2) according to claim 1,
characterized in that
the treatment medium is discharged.

29. The method for disinfecting and/or sterilizing objects (2) according to claim 1,
characterized in that
a neutralizing medium is introduced to neutralize the treatment medium.

30. A software program for carrying out the method according to claim 1.

31. A device (1) for disinfecting and/or sterilizing objects (2), comprising
a process chamber (10) that can be evacuated,
a first connection (110) and/or first reservoir for a treatment medium
a first inlet (100) in the process chamber (10); wherein the first inlet (100) is connected to a first connection (110) and/or the first reservoir for the treatment medium
a second connection (210) and/or second reservoir for a ventilation medium
a second inlet (200) in the process chamber (10), wherein the second inlet (200) is connected to the second connection (210) and/or the second reservoir for the ventilation medium and/or the first connection (110) and/or the first reservoir.
a pump (320) connected to one or more outlets (300)
a control (600) for controlling the first inlet (100), the second inlet (200), the first connection (110), the second connection (210) and/or the outlet (300).
a sensor unit (500).

32. The device (1) for disinfecting and/or sterilizing objects (2) according to claim 31,
characterized in that
the device (1) has an outlet (300) and a medium discharge (330) for discharging the treatment medium from the process chamber (10).

33. The device (1) for disinfecting and/or sterilizing objects (2) according to claim 31,
characterized in that
the control (600) is coupled to the sensor unit (500), the pump (320), the first inlet (100), the second inlet (200) and/or the outlet (300).

34. The device (1) for disinfecting and/or sterilizing objects (2) according to claim 31,
characterized in that
the sensor unit (500) has a sensor (520).

35. The device (1) for disinfecting and/or sterilizing objects (2) according to claim 34,
characterized in that
a sensor (520) of the sensor unit (500) is a pressure sensor.

36. The device (1) for disinfecting and/or sterilizing objects (2) according to claim 35,
characterized in that
the sensor (520) is arranged in the process chamber (10).

37. The device (1) for disinfecting and/or sterilizing objects (2) according to claim 31,
characterized in that
a sensor (530) is a chemical sensor which is suitable for and/or is provided for detecting a chemical measurement variable.

38. The device (1) for disinfecting and/or sterilizing objects according to claim 37,
characterized in that
a sensor (530) is a chemical sensor which is suitable and/or provided for detecting a physical measurement variable from which the concentration of the treatment medium in the process chamber (10) can be determined.

39. The device for disinfecting and/or sterilizing objects according to claim 37,
characterized in that
the chemical sensor (530) is arranged in the process chamber (10) or in the medium discharge (330) for discharging the treatment medium from the process chamber (10).

40. A disinfected object (2) with a surface roughness Rz with Rz<=10 μm, preferably with Rz<=5 μm and particularly preferably with Rz<=1 μm and/or with a porosity with a pore size P with 10 nm<=P<=500 μm, preferably with 25 nm<=P<=100 μm and particularly preferably with 50 nm<=P<=50 μm and/or a pore volume V based on the total volume of the object with V>=70%, preferably V>=55% and particularly preferably V>=40% and with residues of the treatment medium and/or reaction products of the treatment medium of less than 2000 germs per m² on the surface.

41. The disinfected object (2) having an internal surface (23) according to claim 40,
characterized in that
the disinfected object (2) contains residues of peracetic acid or residues of reaction products of peracetic acid.

42. The disinfected object (2) according to claim 40 or 41,
characterized in that
the porosity is an open porosity.

43. A sterilized object (2) having a surface roughness Rz with Rz<=10 μm, preferably with Rz<=5 μm and particularly preferably with Rz<=1 μm and/or with a porosity with a pore size P with 10 nm<=P<=500 μm, preferably with 25 nm<=P<=100 μm and particularly preferably with 50 nm<=P<=50 μm and with residues of the medium and/or reaction products of the medium and/or a pore volume V with V>=70%, preferably V>=55% and particularly preferably V>=40%, based on the total volume of the object, and a germ-free surface.

44. The sterilized object (2) having an internal surface (23) according to claim 43,
characterized in that
the sterilized object (2) has residues of peracetic acid or residues of reaction products of peracetic acid.

45. The sterilized object (2) according to claim 43,
characterized in that
the porosity is an open porosity.
